(19) United States
(12) Patent Application Publication
(10) Pub. No.: US 2021/0205071 A1
(43) Pub. Date: Jul. 8, 2021

(54) INTRAOCULAR LENS INJECTOR WITH PUSH CONTROL DEVICE

(71) Applicant: Andrea Chini, Roma (IT)

(72) Inventor: Andrea Chini, Roma (IT)

(21) Appl. No.: 17/056,793

(22) PCT Filed: May 16, 2019
(86) PCT No.: PCT/IT2019/000038

§ 371 Date: Nov. 19, 2020

(30) Foreign Application Priority Data
May 21, 2018 (IT) 102018000005554

(51) Int. Cl. A61F 2/16

(52) U.S. Cl. CPC A61F 2/167

(57) ABSTRACT

An intraocular lens injector with push control device has, in a pen-like casing (1), a front part (2) including a loading chamber (3), a tapered delivery channel (4) and a distal tip (5), a rear part including an actuator (18) comprising a coil spring (8) acting on a push rod (9) connected anteriorly to an insertion rod (19) for inserting the intraocular lens (IOL) in a patients eye, the coil spring (8) being housed on a spring-holding sleeve (10) provided with a front abutment member (11) joined to the push rod (9), and an intermediate handle part (6) on which a device for controlling the advancement of the push rod (9) is provided. The coil spring (8) has a rear end (13) with a variable longitudinal position by means of a screw and nut screw device (14) that adjusts the loading of the coil spring (8).

### FIELD OF THE INVENTION

The present invention relates to an intraocular lens injector (IOL) with push control device. In general, the injector comprises a front part, having a loading chamber, a tapered delivery channel and a distal tip for exiting the IOL, and a handle portion including an actuator which delivers the IOL through a push rod into the eye of a patient.

### PRIOR ART

US 2014/0257317A1 describes a pen-shaped IOL injector, which includes a loading chamber and a connected delivery tube, and a coil spring-loaded push rod for urging the IOL through the delivery tube, out of a distal tip thereof. The injector includes an actuator that is cocked to compress an automatic delivery coil spring. A braking mechanism allows to control the spring-biased IOL advancement. The force applied by the spring following its loading is not adjustable. The surgeon can only exert an antagonistic action on the advancement of the push rod by means of a pair of arms hinged on the body of the instrument. Each arm has a proximal end that blocks the coil spring, and a distal end interacting by friction on the surface of the push rod.

EP 0 937 443 A2 discloses an intraocular lens injector comprising a pen-like housing, having: a front part including a loading chamber, a tapered delivery channel and a distal tip for exiting the intraocular lens (IOL), a rear part including an actuator comprising a coil spring acting on a push rod connected anteriorly to an insertion rod for inserting the intraocular lens (IOL) into the patient's eye, the coil spring being housed on a spring-holding sleeve provided with a front end connected to the push rod, and an intermediate handle portion on which a device for controlling the advancement of the push rod is provided, whereby the coil spring has a rear end with a longitudinal position variable by means of a screw device which adjusts the loading of the coil spring.

U.S. Pat. No. 2,472,116 relates to a syringe holder which operates a syringe automatically by means of a coil spring loaded to the desired size. However, the adjustment of the spring loading serves to allow the insertion of syringes of different sizes into the syringe holder, or to allow a different stroke of a syringe plunger. In other words, when the amount of liquid to be expelled through the needle is greater, the spring is loaded more. U.S. Pat. No. 2,472,116 has a braking lever mechanism acting on the spring holder by means of a serrated surface.

Turning to the IOL injector, it would be useful for a surgeon to be able to adjust the force exercised on the IOL in the loading chamber depending on the operating conditions.

### SUMMARY OF THE INVENTION

Therefore, an object of the present invention is to adjust the force exercised by the coil spring on the push rod.

A further object of the invention is to provide the injector with small dimensions and an ergonomic shape that facilitates the use thereof. To achieve these and other objects, the invention provides an intraocular lens injector with controlled insertion according to claim 1 and the claims dependent on it.

### BRIEF DESCRIPTION OF THE DRAWINGS

Further features and advantages of the present invention will appear most clearly from the indicative, and therefore non-limiting, description of embodiments of an IOL injector with controlled insertion, as illustrated in the enclosed drawings in which:

FIG. 1 is a schematic longitudinal cross-section view of an IOL injector according to a first embodiment of the invention, in a rest position;

FIGS. 2a to 2e are schematic longitudinal cross-section views similar to that of FIG. 1 and show the injector in successive operating steps;

FIG. 3 is a top view of an IOL injector according to a second embodiment of the invention;

FIG. 4 is an enlarged view of the left end of the IOL injector in FIG. 3;

FIG. 5 is a schematic longitudinal cross-section view of the IOL injector in FIG. 3, in the rest position;

FIG. 6 is an enlarged perspective view of the screw and nut screw device for adjusting the loading of a coil spring;

FIG. 7 is a cross-section view on an enlarged scale along the line A-A in FIG. 5;

FIGS. 8a to 8e are schematic longitudinal cross-section views similar to that in FIG. 5 and show the injector in successive operating steps;

FIGS. 9 to 12 are a perspective view, a front view, a partial perspective longitudinal cross-section view and a plane longitudinal cross-section view respectively, according to a third embodiment of the invention;

FIGS. 13 and 14 are partial enlargements of FIG. 12 in two operating steps of the injector; and

FIGS. 15 and 16 are a plane longitudinal cross-section view and an enlarged transverse cross-section view formed along the line B-B in FIG. 15 respectively, according to a fourth embodiment of the invention.

### DESCRIPTION OF EMBODIMENTS OF THE INVENTION

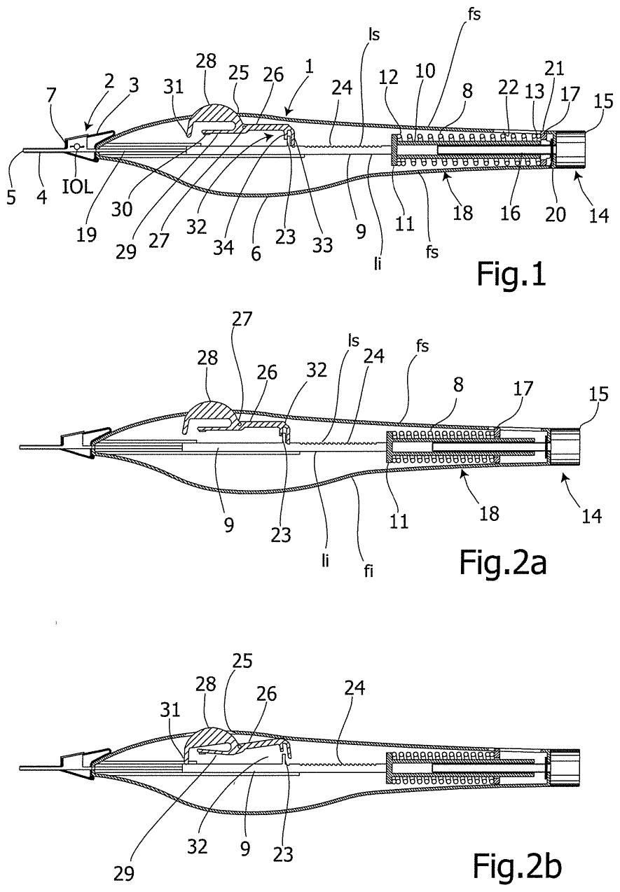

Reference is initially made to FIG. 1, which is a schematic longitudinal cross-section view of a first embodiment of an IOL injector, or simply an injector, according to the invention in a rest position.

The injector comprises a pen-like casing 1, having an upper flank fs and a lower flank fi. The casing 1 has a front part 2 which includes a loading chamber 3, a tapered delivery channel 4 and a distal tip 5 for exiting an intraocular lens, which is indicated with IOL and shown schematically in the loading chamber 3. One front door 7 allows the introduction of the IOL into the loading chamber 3.

The casing 1, which has an intermediate handle part 6, includes at the rear an actuator 18 provided with a coil spring 8 for automatic delivery of the IOL into the patient's eye by means of a push rod 9 bearing frontally an insertion rod 19 for moving the IOL.

According to the present invention the push rod 9 is of a prismatic shape, having an upper side ls and a lower side li:

The coil spring 8 is housed externally concentrical to an internally smooth spring-holding sleeve 10. The spring-holding sleeve 10 has a front abutment member 11 joined to the push rod 9. The coil spring 8 has a front end 12 abutted against the front abutment member 11 and a rear end 13 that has a variable longitudinal position by means of a screw and nut screw device 14 which adjusts the loading of the coil spring 8.

The screw and nut screw device 14 comprises an adjustment knob 15, located in the rear end 20 of the casing 1 of the injector. The adjustment knob 15 is provided with a threaded shaft 16 freely inserted in the spring-holding sleeve 10. Mounted on the threaded shaft 16 is a nut 17 for abutting the rear end 13 of the coil spring 8. The nut 17 is in a screw and nut screw coupling with the threaded shaft 16. A detailed representation of the screw and nut screw device 14 is described below, with reference to a second embodiment of the invention.

The abutment nut 17 of the coil spring 8 has at least one projecting index 21. The casing 1 of the injector has at least one longitudinal slot 22 able to allow the protruding index 21 to slide along the slot 22 itself.

A graduated scale, which will be shown later, is provided on the casing 1 of the injector adjacent to the slot 22 for reading the position of the rear end 13 of the coil spring 8 by means of the projecting index 21 of the abutment nut 17 for the rear end of the coil spring 8.

The push rod 9 has, on its upper side Is, a projection 23 projecting upwards and a toothed rack 24 also facing upwards.

A push rod advancement control device is provided, which is described below.

The push rod advancement control device comprises a lever which acts on the toothed rack 24. The lever has a first arm, on which is the applied force, in the form of a button 28 protruding from the upper flank fs of the casing 1 through a cutout 25, and a second arm 29, on which is the resistant force, resiliently deformable in abutment with the casing 1. The second arm 29 acts as a returning elastic member for the button 28. A fulcrum 27 is located between the first arm and the second arm of the lever inside the casing.

The second arm 29 of the lever is in front of the button 28 and extends rearwardly to a free end 32 that is provided with a pair of fingers 33, 34 facing downwards for locking the projection 23 projecting upwards in the push rod 9 and engaging a tooth of the toothed rack 24. Furthermore, the button 28 has a front end 31 facing downwards for frictional engagement with the push rod 9.

In greater detail, the casing 1 has the cutout 25 in its upper flank fs. The second arm 29 of the lever is extended in an arm 26 from the fulcrum 27. The arm 26 is joined to the button 28. The button 28 projects upwards through the cutout 25 to allow a manual control.

The second lever arm 29 is elastically deformable for the abutment with a fixed internal part of the casing indicated with 30.

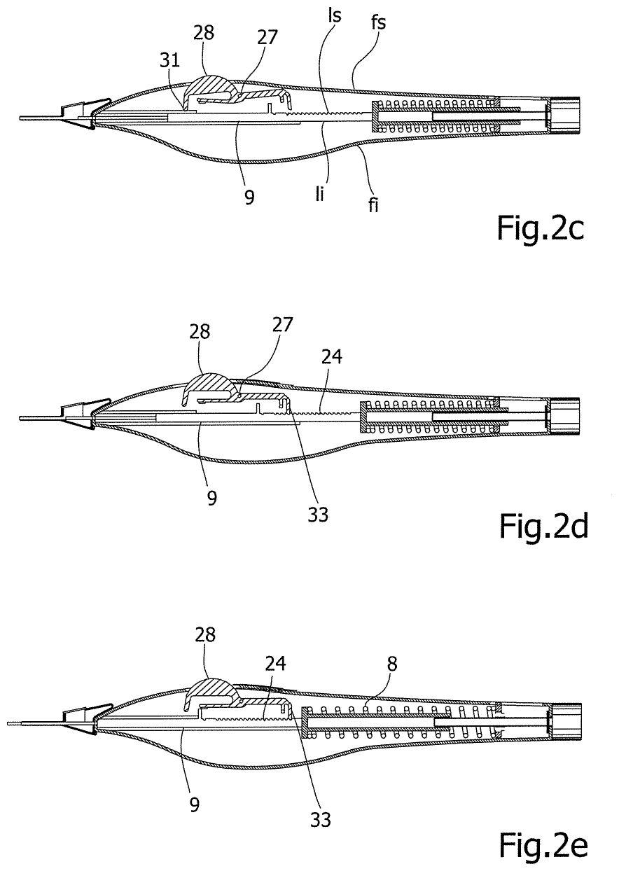

The button 28 has a front end 31 facing downwards due to the frictional engagement with the push rod 9. The arm 26 has a rear grip end 32, which is provided with a pair of fingers 33, 34 facing downward. Together, the fingers 33, 34 block the projection 23 projecting upwards in the push rod 9, while the single finger 33, longer than the finger 34, engages the teeth 24 of the push rod 9 to obtain a rapid locking thereof, as shown in FIGS. 2a to 2e. They are schematic longitudinal cross-section views similar to those in FIG. 1 showing the injector in subsequent operating steps. Shown in FIG. 2a are the button 28 in the rest position, and the rear end 32 of the arm 26, to which the button 28 is joined. The rear end 32 is engaged to retain the projection 23 projecting upwards. In this way, the push rod 9 is kept still. By acting on the adjustment knob 15 of the screw and nut screw device 14 the spring 8 is loaded that is abutted between the front abutment member 11 and the rear abutment nut 17. In this step the injector is loaded and ready for the use, the force that must be applied by the spring to insert the IOL into the patient's eye having been adjusted.

FIG. 2b shows that the surgeon has pressed the button 28 against the antagonistic action of the elastically deformable second lever arm 29 and released the projection 23 projecting upwards in the push rod 9 from the engagement with the rear end of socket 32 of the arm 26. The front end 31 of the button 28 is in friction contact with the push rod 9 to control its advancement.

In FIG. 2c it is shown that the push rod 9 is advanced thanks to an action on the button 28, which, when compressed, presses by friction its tip 31 on the push rod 9, adjusting its advancement.

FIG. 2d shows that the release of the button 28 stops the advancement of the push rod 9, thanks to the engagement of the finger 33 with the teeth 24 protruding upwards.

Finally, in FIG. 2e it is shown that the button 28 has been pressed slightly to disengage the finger 33 from the teeth 24 for the final displacement of the push rod 9 under the effect of the spring 8, now completely relaxed.

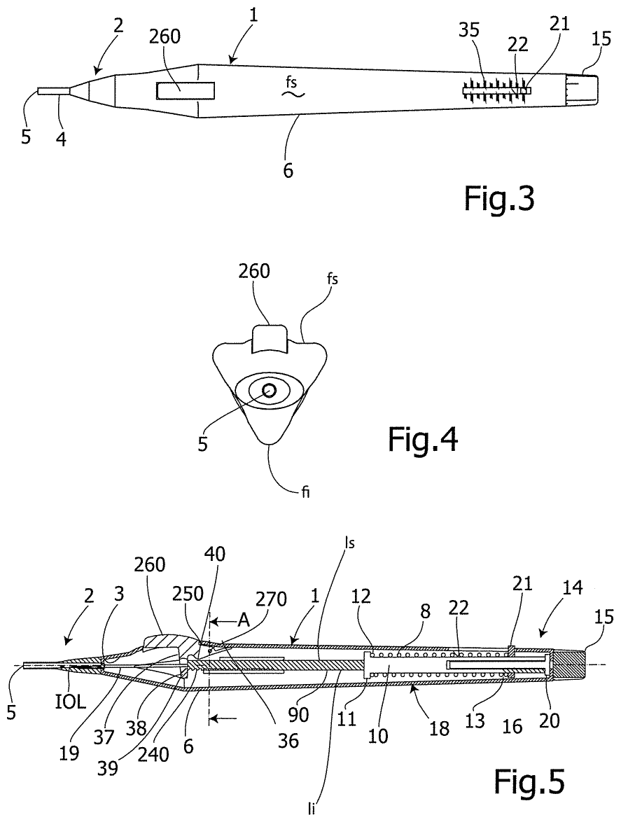

Reference is made now to FIGS. 3 to 5, which are a top view, an enlarged left end view and a schematic longitudinal cross-section view, respectively, of the IOL injector according to a second embodiment of the invention. Parts that are the same or similar to those in the first embodiment are marked with the same reference numerals. Differences concern the push rod advancement control device.

Like in the first embodiment, the injector comprises a casing 1, having a front part 2, a tapered delivery channel 4 and a distal tip 5 for exiting an IOL.

The casing 1, which has an intermediate handle part 6, includes at the rear an actuator 18 provided with a coil spring 8 for automatically delivering the IOL into the patient's eye by means of a push rod 90, frontally bearing an insertion rod 19 for the displacement of the IOL. As shown in FIG. 7, the push rod has a prismatic shape.

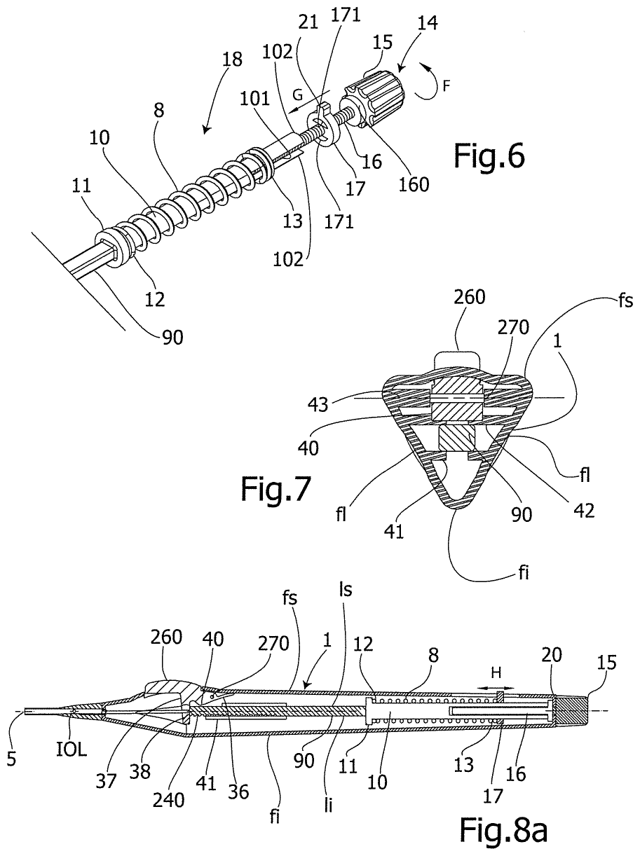

The actuator 18 has a screw and nut screw device 14 for adjusting the loading of the coil spring 8. As shown in FIG. 6, which is an enlarged perspective view of the screw and nut screw device 14, the coil spring 8 is housed externally concentric to the internally smooth spring-holding sleeve 10. The spring-holding sleeve 10 has a front abutment member 11 joined to the push rod 90. The coil spring 8 has a front end 12 abutted to the front abutment member 11 and a rear end 13 which has a variable longitudinal position by means of the screw and nut screw device 14 which adjusts the loading of the coil spring 8. The spring-holding sleeve 10 has a rear portion lightened with two opposite grooves 101 so that the spring-holding sleeve 10 is reduced to two opposite upper and lower profiles 102, having basically a half-moon cross-section.

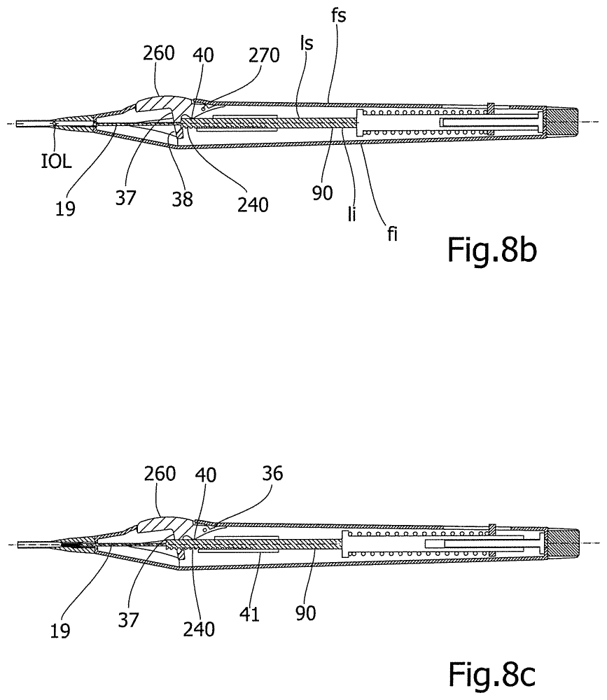

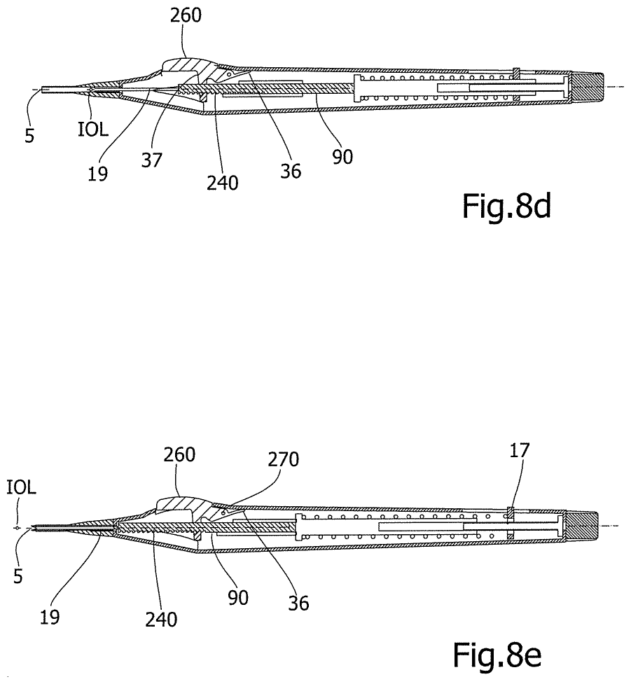

The screw and nut screw device 14 comprises an adjustment knob 15, located beyond the rear end 20 of the injector. The adjustment knob 15 is only allowed to rotate, for example in the direction of the arrow F (FIG. 6) thanks to an abutment 160 which abuts the rear end 20 of the injector casing (FIG. 8a). The adjustment knob 15 is provided with a threaded shaft 16 freely inserted in the spring-holding sleeve 10. Mounted on the threaded shaft 16 is a nut 17 abutting the rear end 13 of the coil spring 8.

The nut 17 is internally threaded to engage the threaded shaft 16.

The nut 17 has two opposed openings 171 matching the opposite upper and lower profiles 102 of the spring-holding sleeve 10 so that the nut 17 can advance according to the arrow G against the spring end 13 when the knob 15 is rotated in the direction of the arrow F. In FIG. 6, for convenience of representation only, the nut 17 is shown separated from the spring-holding sleeve 10 on which the nut 17 is actually mounted to allow it to advance. As already mentioned for the first embodiment, the nut 17 has a projecting index 21 (FIG. 3) movable inside the longitudinal slot 22 to display the adjustment of the loading of the spring 8.

For this purpose, a graduated scale is shown in FIG. 3 and indicated as 35. The graduated scale 35 could also be provided on the opposite flank of the casing near another longitudinal slot 22 for advancing another index 21.

The push rod 90 has a lower side provided with a toothed rack 240.

A device is provided for controlling the advancement of the push rod, which differs from that of the first embodiment as described below.

The casing 1 has a cutout 250.

The device for controlling the advancement of the push rod 90 comprises a lever which acts on the toothed rack 240. The lever has a first arm, on which is the applied force, in the form of a button 260 projecting from the upper side fs of the casing 1 through the cutout 250, and a second arm 36, on which is the resisting force, elastically deformable in abutment with the casing 1, in particular its upper side fs. A fulcrum 270 is located between the first arm and the second arm of the lever inside the casing 1.

The button 260 has a protrusion 37 which extends downwards and is provided with a through hole 38 to be crossed by the push rod 90, as well as a pawl 39 engaging the toothed rack 240, and a side-by-side convex portion 40 for engaging by friction the upper side Is of the push rod 90.

More in detail, the push rod advancement control device includes the button 260 hinged inside the casing in a fulcrum 270 and protruding through the cutout 250. The button 260 has a small plate as a second lever arm 36, which extends rearwardly to the fulcrum 270 and is elastically deformable for the abutment of the button 260 with the casing 1. Furthermore, the button 260 has the protrusion 37 facing downwards, equipped with a through hole 38 for the push rod 90, which engages the toothed rack 240. The button 260 has, next to the protrusion 37, a convex portion 40 engaging by friction the push rod 90 on the opposite side to the toothed rack 240.

With reference to FIG. 7, which is a cross-section on an enlarged scale according to the line A-A of FIG. 5, it is shown how the button 260 is mounted inside the injector casing 1. The push rod 90 rests slidingly on longitudinal supporting walls 41, 42, while the longitudinal wall 43 has the purpose of supporting the push-button 260, which is hinged with the fulcrum 270.

Reference is now made to FIGS. 8a to 8e which are schematic longitudinal cross-section views similar to those of FIG. 5 and show the injector in successive operating steps. FIG. 8a shows that the button 260 is in a rest position and blocks the advancement of the push rod 90. Thanks to the adjustment knob 15, it is possible to move the nut 17, as indicated by the double arrows H, to adjust the loading of the spring 8, as described with reference to FIG. 6. By acting on the adjusting knob 15 of the screw and nut screw device 14, the coil spring 8 is loaded, which is abuted between the front abutment member 11 and the rear abutment nut 17 of the coil spring 8. In this step, the injector is charged and ready for use, the force that must be applied by the spring to insert the IOL into the patient's eye having been adjusted.

FIG. 8b shows that the surgeon has pressed the button 260 against the antagonistic action of the elastically deformable plate 36, so as to position its own protrusion 37 with the through hole 38 facing the push rod 90.

In FIG. 8c it is shown that the push rod 90 is advanced thanks to an action described above on the button 260. The convex portion 40 of the button 260 presses by friction on the push rod 90, on the opposite side to the toothed rack 240, to adjust the advancement thereof.

FIG. 8d shows that the release of the button 260 stops the advancement of the push rod 90 due to the engagement of the protrusion 37 with the succession of the teeth 240 projecting downwards.

Finally, in FIG. 8e it is shown that the button 260 has been pressed slightly to disengage the protrusion 37 from the teeth 240 for the final displacement of the push rod 90 under the effect of the spring 8, now completely relaxed. The IOL is shown anteriorly to the distal end 5 thus exiting into the patient's eye.

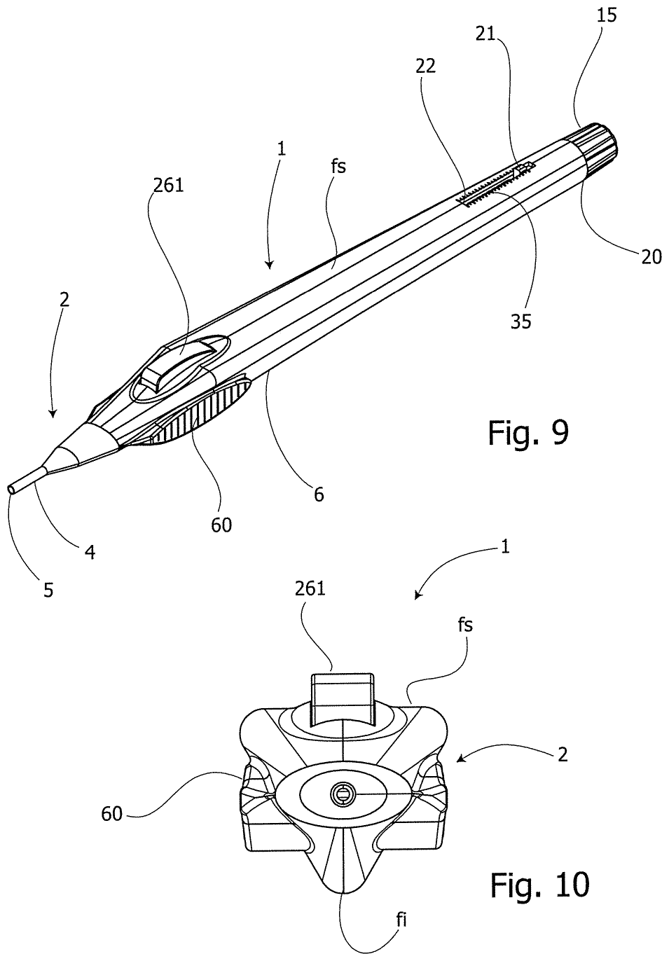

Reference is made now to FIGS. 9 to 12, which are a perspective view, a front view, a perspective longitudinal cross-section view and a plane longitudinal cross-section view, respectively, according to a third embodiment of the IOL injector of the present invention. In FIG. 12 a plurality of small circles show the assembly points of two halves of the casing 1.

As seen later, differences concern both the actuator with a coil spring acting on the push rod, and the push rod control advancement device. Same or similar parts to those in the second embodiment are designated with the same reference numerals.

Like in the previous embodiments, the injector comprises a pen-like casing 1, having a front part 2 which includes a loading chamber 3, a tapered delivery channel 4 and a distal tip 5 for exiting an intraocular lens.

The casing 1 has a central grip part 6, with opposed knurled portions 60 to improve the operator's grip. The casing 1 includes at the rear an actuator 180 provided with a coil spring 8 for automatically delivering the IOL into the patient's eye by means of a push rod 900, frontally bearing the insertion rod 19 for displacing the IOL. The push rod 900 has a prismatic shape.

The coil spring 8 is housed externally concentrical with an internally smooth spring-holding sleeve 100. The spring-holding sleeve 100 is shorter than in the previous embodiments, not having that rear portion lightened with two opposed grooves which, in the previous embodiments, allow the engagement between the nut 17 acting as a nut screw and the threaded spindle 16 operating as a screw in the screw and nut screw device 14 of the actuator 18.

The spring-holding sleeve 100 has its front abutment member 11 joined to the push rod 9. The coil spring 8 has its front end 12 abutted against the front abutment member 11 and its rear end 13 that has a variable longitudinal position by means of a screw and nut screw device 140 that adjusts the loading of the coil spring 8.

The screw and nut screw device 140 includes the adjustment knob 15, located beyond the rear end 20 of the injector casing 1. The adjustment knob 15 is provided with a shaft 70 having an abutment 160 which abuts the rear end 20 of the injector casing. The shaft 70, which is cylindrical, has a proximal threaded portion 161 and a smooth distal portion 162. The distal portion 162 is freely inserted in the spring-holding sleeve 100. On the proximal threaded portion 161 of the shaft 70 is engaged, in coupling of screw and nut screw, a nut 170 abutting the rear end 13 of the coil spring 8. The abutment nut 170 of the coil spring 8 has a pair of opposite indexes 21. The injector casing 1 has two longitudinal slots 22 able to allow the protruding index 21 to slide along the slot 22 itself.

A graduated scale is provided on the injector casing 1 adjacent to the longitudinal slot 22 for reading the position of the rear end 13 of the coil spring 8 by means of the protruding index 21 of the nut 170 for rear abutting the coil spring 8.

Unlike the previous embodiments, the push rod 900, which has a prismatic shape, has a lower side li with a succession of lower teeth 240 and an upper side Is with a succession of upper teeth 241.

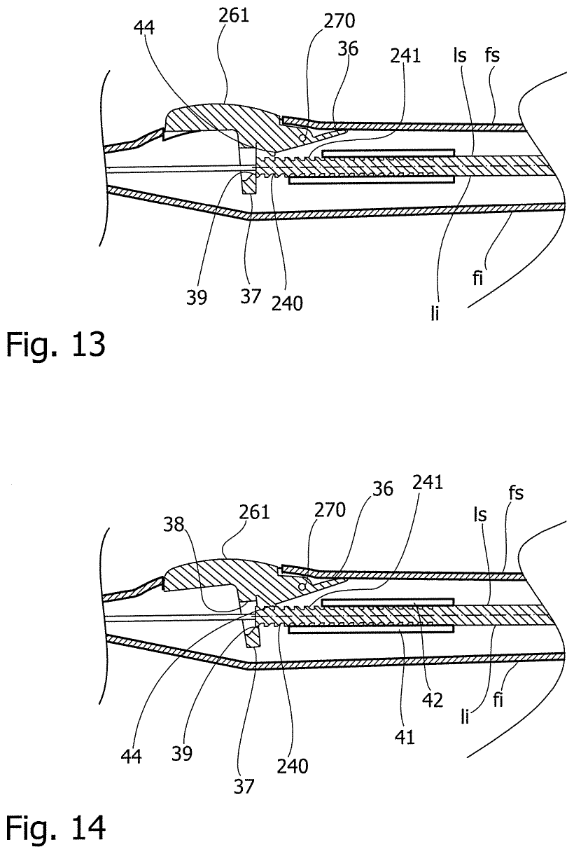

Co-operating with these successions of teeth is a device for controlling the advancement of the push rod cooperates, which differs from that of the second embodiment, as described below with reference to FIGS. 13 and 14 which are partial enlargements of FIG. 12 in two moments of the injector operation.

Like in the second embodiment, the device for controlling the advancement of the push rod 900 comprises a lever which acts, however, on the toothed rack 240, 241. The lever has a first arm, on which is the applied force, in the in the form of a button 261 projecting from the upper flank fs of the casing 1 through the cutout 250, and a second arm 36, on which is the resisting force, that is elastically deformable in abutment with the casing 1. The fulcrum 270 is located between the first arm and the second arm of the lever inside the casing 1.

The button 261 has a protrusion 37 which extends downwards and is provided with a through hole 38 to be traversed by the push rod 900, as well as a pawl 39 engaging the toothed rack 240, and a side-by-side convex portion 44 engaging the toothed rack 241 on the upper side ls of the push rod 900.

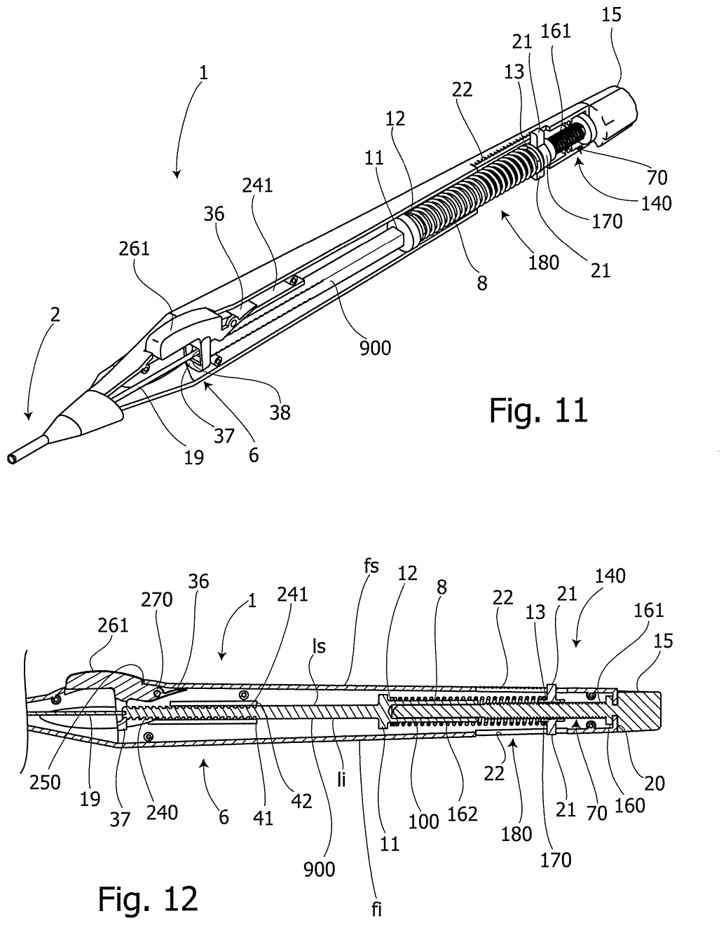

In the cutout 250 of the casing 1 a button 261 is hinged inside the casing in a fulcrum 270 but protruding through the cutout 250. The button 261 has a small plate 36, which extends behind the fulcrum 270, which is elastically deformable for the abutment of the button 261 with the casing 1. As best shown in FIG. 11, the button 261 has a downwardly protrusion 37 provided with a through hole 38 to be traversed by the push rod 900. The protrusion 37 engages, thanks to its through hole 38, with the teeth of the succession of lower teeth 240 by means of a projection 39. The upper teeth 240, cooperating with the projection 39 of the protrusion 37 function as teeth for a rapid block of the advancement, as shown in FIG. 14. On the opposite side, the toothed rack 241 faces a convex portion 44 adjacent to the projection 37 downwards. Thanks to the mutual engagement of the teeth 241 with the convex portion 44, it is possible to obtain a reduction in the speed of advancement of the push rod 900 or possibly a stop thereof. In other words, with reference to FIG. 13, it is shown that the button 261 is in a raised position thanks to the contrasting action of the elastic plate 36; that is, the button 261 is not pressed downwards by a pressure, for example of the thumb, applied to it. In this arrangement, it is noted that the projection 39 acting on the lower teeth 240 blocks the advancement of the push rod 900. When the push-button 261 is pressed downwards as shown in FIG. 14, the projection 39 disengages the lower teeth 240, but the convex portion 44 begins to engage the upper teeth 241 to create a slowing of the advancement of the push rod 900 or a stop thereof.

In this way, the advance control device allows to adapt the IOL delivery speed depending on the operating conditions of the moment.

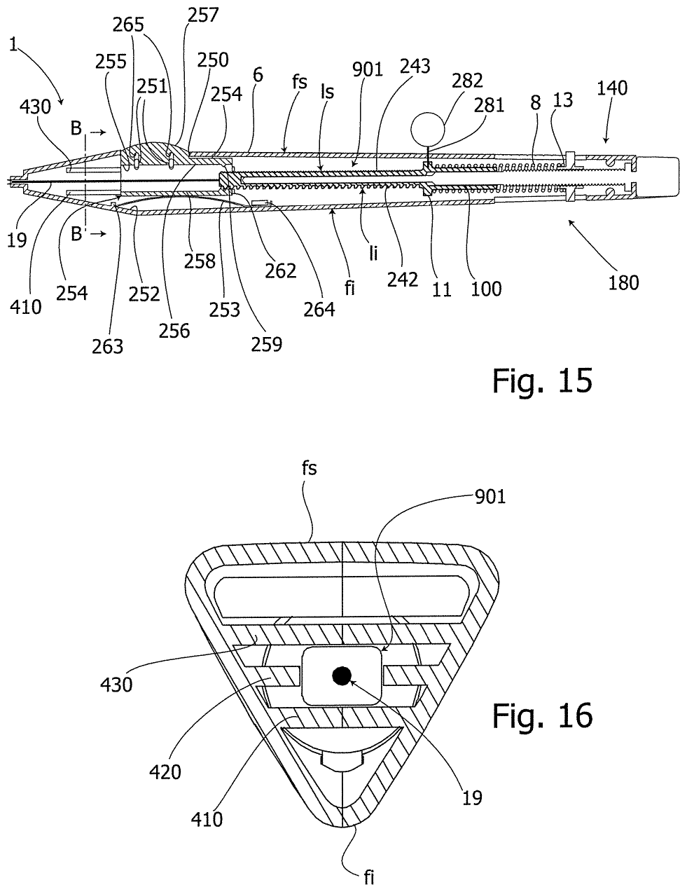

Reference should now be made to FIGS. 15 and 16, which are a plane longitudinal cross-section view and a transverse cross-section view obtained along the line B-B in FIG. 15, respectively, according to a fourth embodiment of the IOL injector according to the present invention.

As seen later, differences concern both the actuator with a coil spring acting on the push rod and the push rod advancement control device. Same parts or similar to those in the third embodiment are designated with the same reference numerals.

Provided on the casing 1 is a through hole, and a stop element traverses the through hole to engage the push rod 901 in a waiting condition of the injector prior to its operation.

The front abutment member 11 joined to the push rod 901 has a circumferential recess. The stop element is a pin 281 provided with a ring 282 at its outer end and a tip at its inner end. The tip is designed to be received in the circumferential recess of the front abutment member 11.

The stop element is useful to prevent an involuntary actuation of the push rod advancement control device can move the latter before the surgeon is ready to voluntarily operate the injector.

The thrust element 901, preferably of a prismatic shape, has a toothed rack 242 on the lower side li of the push rod 901 and a knurled tooth rack 243 on the upper side ls of the push rod 901.

The casing 1 has, internally integral with its lateral flanks fl, longitudinal supporting walls 410, 420, 430 acting as a guide for the push rod 901 in its longitudinal sliding.

Furthermore, the casing 1 internally contains at least one pair of transverse pins 251 integral with at least one side flank fl of the casing 1. The pair of transverse pins 251 can be not integral with both the side flanks fi of casing 1 if the casing 1 is made of two symmetrical halves connected each other. The pair of transverse pins 251 is positioned below the cutout 250. On the side opposite to the cutout 250, formed on the lower side fi of the casing 1 is a spring seat 252. In the seat 252 a leaf spring 253 is positioned: one front end thereof abuts to a projection 263, and a rear end thereof is slidably received under a guide 264.

According to this fourth embodiment of the invention, the advancement control device of the push rod 901 comprises a tubular element 254 provided with an internal cavity 255 for the passage of the push rod 901. The tubular element 254 has an upper wall 256 with a button-shaped bulge 257 protruding at the top from the cutout 250 formed in the upper flank fs of the casing 1. The button-shaped bulge 257 has two through slots 265 that receive the transverse pins 251: this arrangement causes the vertical sliding of the button-shaped bulge 257 through the cutout 250. Therefore, the tubular element 254 is transversally slidable in the casing 1. The tubular element 254 has a lower wall 258 operating as a ledge for the leaf spring 253 received in the seat 252 of the casing 1.

The upper wall 256 of the tubular element 254 has a surface facing the internal cavity 255 provided with a wedge-shaped projection 259 adapted to engage the succession of knurled teeth 243 on the upper side ls of the push rod 901.

The lower wall 258 of the tubular element 254 has a surface facing towards the internal cavity 255 provided with at least a pair of teeth 262 able to engage with the toothed rack 242 on the lower side of the push rod 901.

The operation of the push rod advancement control device according to the fourth embodiment of the invention provides that the tubular element 254 engages the toothed rack 242 in a rest condition, for example before using the injector. This is guaranteed by the fact that the return spring 253 pushes the tubular element 254, constrained to slide only vertically, in its upper position in which its teeth 262 are engaged with the teeth 242 of the push rod. Once the retaining element 281 of the push rod 901 has been released, the thrust element would advance under the effect of the coil spring 8, but the wedge-shaped projection 259 on the inner surface of the upper wall 256 of the tubular element 254 engages the knurled tooth rack 243 on the upper side Is of the push rod 901, controlling the advancement of the latter. It should therefore be understood that the push rod 901 is never free under the effect of the coil spring 8, but it is under the control of the tubular element 254 pushed by the surgeon thanks to its bulge 257 protruding from the cutout 250.

It should be understood that, thanks to the various embodiments of the invention, the object thereof has been achieved. The invention's scope is defined by the enclosed claims.

1. An intraocular lens injector with push control device comprising a pen-like casing (1) having:
a front part (2) including a loading chamber (3), a tapered delivery channel (4) and a distal tip (5) for exiting the intraocular lens (IOL),
a rear part including an actuator (18; 180) comprising a coil spring (8) acting on a push rod (9; 90; 900; 901) connected anteriorly to an insertion rod (19) for inserting the intraocular lens (IOL) in a patient's eye, the coil spring (8) being housed on a spring-holding sleeve (10; 100) provided with a front abutment member (11) joined to the push rod (9; 90; 900; 901), and
an intermediate handle part (6) on which a device for controlling the advancement of the push rod (9; 90; 900; 901) is provided;
characterized in that the coil spring (8) has a rear end (13) with a variable longitudinal position by means of a screw and nut screw device (14; 140) that adjusts the loading of the coil spring (8).

2. The injector according to claim 1, wherein the spring holder sleeve (10) is internally smooth and provided with rear ends (102) with a crescent-shaped profile, and the screw and nut screw device (14) comprises an adjustment knob (15) that is located in the rear end (20) of the pen-like casing (1) and provided with a threaded shaft (16) inserted in the spring holder sleeve (10) and provided with an abutment nut (17) for the rear end (13) of the coil spring (8), the abutment nut (17) being mounted in engagement with the threaded shaft (16) and being provided with crescent-shaped slots (171) for sliding engagement with the rear ends (102) of the spring holder sleeve (10).

3. The injector according to claim 2, wherein the abutment nut (17) of the coil spring (8) has a protruding index (21), and the pen-like casing (1) has a longitudinal slot (22) suitable for allowing the protruding index (21) to slide along the longitudinal slot (22).

4. The injector according to claim 1, wherein the spring holder sleeve (100) is internally smooth, and the screw and nut screw device (140) comprises an adjustment knob (15) that is located in the rear end (20) of the pen-like casing (1) and provided with a threaded shaft (70) having an abutment (160) abutting the rear end (20) of the pen-like casing (1), a proximal threaded portion 161 and a smooth distal portion 162 freely inserted in the spring-holding sleeve 100, on the proximal threaded portion 161 of the shaft 70 being engaged, in coupling of screw and nut screw, a nut 170 abutting the rear end 13 of the coil spring 8.

5. The injector according to claim 4, wherein the abutment nut (170) of the coil spring (8) has a pair of opposite protruding indexes (21), and the injector casing (1) has two longitudinal slots (22) able to allow the respective protruding index (21) to slide along the longitudinal slot (22).

6. The injector according to any one of claims 3 and 5, wherein a graduated scale (35) is provided on the pen-like casing (1) adjacent to the longitudinal slot (22) for reading the position of the rear end (13) of the coil spring (8) by means of the protruding index (21) of the abutment nut (17; 170) abutting the rear end (13) of the coil spring (8).

7. The injector according to claim 1, wherein a through hole is provided on the pen-like casing (1), and a stop element traverses the through hole to engage the push rod (901) in a waiting condition of the injector preceding its operation.

8. The injector according to claim 7, wherein the front abutment member (11) joined to the push rod (901) has a circumferential recess, and the stop element is a pin (281), provided with a grip ring (282) at its outer end, and with a tip at its inner end intended to be received in the circumferential recess of the front abutment member (11).

9. The injector according to claim 1, wherein the pen-like casing (1) has internally integral with at least one of the lateral flanks (fl) thereof longitudinal supporting walls (41, 42; 410, 420, 430) acting as a guide for the push rod (90; 900; 901) in its longitudinal sliding.
